(19) United States
(12) Patent Application Publication
(10) Pub. No.: US 2018/0271406 A1
(43) Pub. Date: Sep. 27, 2018

(54) Combined Sensor Apparatus for Breath Gas Analysis

(71) Applicants: Spirosure, Inc., Pleasanton, CA (US); NGK Spark Plug Co, Ltd., Nagoya (JP)

(72) Inventors: Keizo Furusaki, Nagoya (JP); Ryosuke Furuhashi, Komaki (JP); Miyuki Tachi, Ichinomiya (JP); Ryan Leard, Oakland, CA (US); Solomon Ssenyange, Fremont, CA (US)

(73) Assignees: Spirosure, Inc., Pleasanton, CA (US); NGK Spark Plug Co, Ltd., Nagoya (JP)

(21) Appl. No.: 15/936,268

(22) Filed: Mar. 26, 2018

Related U.S. Application Data
(60) Provisional application No. 62/477,395, filed on Mar. 27, 2017.

(51) Int. Cl. A61B 5/08; A61B 5/097; A61B 5/00

(52) U.S. Cl. CPC A61B 5/082; A61B 5/0075; A61B 5/097

(57) ABSTRACT

A monitoring system is disclosed that includes features for detecting the presence of biomarkers from a gas sample, such as exhaled breath. An assembly may also includes a plurality of sensors to detect biomarkers present in exhaled breath that are associated with respiratory illness. The biomarkers include carbon dioxide, carbon monoxide, and nitric oxide.

### CROSS REFERENCE TO RELATED APPLICATIONS

This application claims the benefit of U.S. Provisional Application No. 62/477,395 filed on Mar. 27, 2017, the subject matter of which is herein incorporated by reference in its entirety.

### FIELD OF THE INVENTION

The present invention relates generally to monitoring devices used for breath gas analysis, and more particularly to monitoring devices that may be used to test for biomarkers associated with medical conditions, such as chronic obstructive pulmonary disease, pulmonary disease, asthma, and other respiratory diseases.

### BACKGROUND

Breath gas analysis can provide a method of providing information regarding the clinical state of an individual. Typically, a patient provides a breath sample generated from the act of exhalation, and one or more tests is performed on the exhaled breath gas sample. Breath gas analysis can be used to detect a wide range of compounds that are present in the blood and associated with certain medical conditions.

Respiratory diseases are among the most common disorders in the world. Such respiratory diseases include conditions such as chronic obstructive pulmonary disease (COPD), asthma, cystic fibrosis and pulmonary fibrosis. COPD, for example, affects millions of people and is responsible for extensive morbidity and mortality in the United States. COPD is a term used to describe chronic lung diseases characterized by progressive development of airflow limitation that is usually not fully reversible with medication. The common symptoms of COPD include breathlessness, wheezing and a chronic cough. During metabolism, the body uses oxygen and produces carbon dioxide (CO₂), which may be removed from the body through the lungs when a person breathes out. Individuals with COPD can sometimes have excessive CO₂ levels in the blood, an indication that the lungs may be obstructed or blocked, making it more difficult to breathe.

Asthma is another example of a chronic lung disease with symptoms similar to COPD, such as breathlessness and wheezing, but etiologically distinct from COPD. Asthma is a prevalent health care problem; it affects millions in the United States and around the world. A significant fraction of patients with asthma can be classified as having moderate to severe asthma and would benefit from more frequent monitoring of their airway inflammation. Although COPD and asthma require different treatments, test results for COPD and asthma often overlap.

Asthma in particular is characterized by an inflammatory reaction in hyper-reactive airways that restrict airflow into the lungs. In recent years, measurement of exhaled nitric oxide (eNO) has been shown to be a non-invasive and complementary tool to other pulmonary function tests in assessing airway inflammation, specifically in subjects with asthma. Accordingly, the presence of eNO has become a well-known, globally accepted biomarker for airway inflammation.

An effective eNO test would be complimentary to the standard tests, but there is a dearth of inexpensive sensors capable of detecting the minute amounts of nitric oxide (NO) (typically measured in parts per billion) present in exhaled air. Moreover, NO sensors need to provide an accurate NO measurement in the presence of other possibly interfering gas components, including water and CO₂.

Thus, it would be desirable and advantageous to provide an accurate and efficient respiratory monitor capable of conducting tests for biomarkers, and in some instances, multiple biomarkers that are associated with a particular condition or disease. It would also be desirable and advantageous to provide multiple sensors to detect diagnostic markers in an exhaled breath sample in a single unit or apparatus. In some instances, it also may be desirable and advantageous to provide a monitor with a compact and portable footprint that may be useful in a variety of settings, including in mobile health applications. Additionally, it would be desirable and advantageous to provide a detection system that is capable of detecting low concentrations of specific gases with high sensitivity, while discriminating against the various other molecules that may be present in human breath.

### BRIEF SUMMARY OF THE INVENTION

The present invention describes apparatuses with sensors that can detect one or more gases in exhaled human breath. The present invention also describes apparatuses with a combination of sensors that can detect multiple gases in exhaled human breath.

In one embodiment, a multi-sensor apparatus for use in the monitoring of respiratory disease is described. The apparatus comprises a housing comprising a breath flow pathway within the housing; a breath inlet positioned at an entrance of the breath flow pathway; a plurality of sensors positioned in the breath flow pathway, downstream from the breath inlet, wherein each sensor is configured to detect the presence of a biomarker indicative of respiratory disease; and a breath outlet positioned at an exit of the breath flow pathway. In some embodiments, the biomarker may be selected from the group consisting of carbon dioxide (CO₂), carbon monoxide (CO), and nitric oxide (NO). Each sensor may be selected from the group consisting of a CO₂ selective sensor, a CO selective sensor, and an NO selective sensor. The plurality of sensors may comprise a CO₂ selective sensor, a CO selective sensor, and an NO selective sensor.

In another embodiment, a method for monitoring respiratory disease is also described. The method comprises the steps of: flowing a breath gas sample into a housing comprising a breath flow pathway within the housing; flowing the breath gas sample through a breath inlet positioned at an entrance of the housing; exposing at least a portion of the breath gas sample to a plurality of sensors positioned in the breath flow pathway, downstream from the breath inlet; and releasing at least a portion of the breath gas sample through a breath outlet positioned at an exit of the breath flow pathway. Each sensor is configured to detect the presence of a biomarker indicative of respiratory disease.

In addition, an apparatus for detecting respiratory disease comprises a housing, a breath gas inlet, a CO₂ sensor element, a CO sensor element, a NO sensor element, and a breath outlet. The housing comprises a breath flow pathway disposed within the housing. The breath gas inlet is positioned at an entrance of the breath flow pathway. The CO₂ sensor element is positioned in the breath flow pathway, downstream from the breath inlet. The CO sensor element is positioned in the breath flow pathway, downstream from the CO₂ sensor element. The NO sensor element is positioned in the breath flow pathway, downstream from the CO sensor element. The breath outlet is positioned at an exit of the breath flow pathway, downstream from the sensor elements.

In some embodiments, the NO sensor element comprises a micro-channel reactor filter comprising platinum and zeolite, and a potentiometric gas sensor. The NO sensor element may further comprise a micro-channel reactor filter heater relay. The apparatus may include a humidity controller configured to regulate the humidity of a breath sample in at least a portion of the breath flow pathway. The apparatus further may include an excess exhaust portal upstream from the CO₂ sensor element, wherein the excess exhaust portal is adapted to release from the housing a portion of a breath gas sample entering the breath gas inlet.

Also described is a method for monitoring respiratory disease that includes the steps of flowing a breath gas sample through a breath inlet positioned at an entrance of a housing and exposing the breath gas sample to a CO₂ sensor element positioned in the breath flow pathway, downstream from the breath inlet. The housing comprises a breath flow pathway disposed within the housing. The method also includes the step of exposing the breath gas sample to a CO sensor element positioned in the breath flow pathway, downstream from the CO₂ sensor element, and exposing the breath gas sample to a NO sensor element positioned in the breath flow pathway, downstream from the CO sensor element. The breath gas sample is released through a breath outlet positioned at an exit of the breath flow pathway.

In another embodiment, an apparatus for detecting respiratory disease comprises a housing, a breath gas inlet, a CO sensor detection element, a CO₂ sensor detection element, and a breath outlet. The housing comprises a breath flow pathway disposed within the housing. The breath gas inlet is positioned at an entrance of the breath flow pathway. The CO sensor detection element is positioned in the breath flow pathway, downstream from the breath inlet. The CO₂ sensor detection element is positioned in the breath flow pathway, downstream from the CO sensor detection element. The breath outlet is positioned at an exit of the breath flow pathway, downstream from the sensor elements. The apparatus may further include a heating element disposed on an upstream side of the CO sensor detection element. In some embodiments, the CO sensor detection element may include a metal oxide semiconductor type sensor. The CO₂ sensor detection element may include a spectroscopic nondispersive infrared (NDIR) CO₂ sensor.

Further described is a method for detecting respiratory disease that includes the steps of flowing a breath gas sample through a breath inlet positioned at an entrance of a housing; exposing the breath gas sample to a CO₂ sensor detection element positioned in the breath flow pathway, downstream from the breath inlet; and exposing the breath gas sample to a CO sensor detection element positioned in the breath flow pathway, downstream from the CO₂ sensor detection element. The breath gas sample is released through a breath outlet positioned at an exit of the breath flow pathway.

In yet another embodiment, a multi-sensor apparatus for monitoring respiratory disease is provided. The apparatus comprises a housing comprising a breath flow pathway disposed within the housing and a breath inlet positioned at an entrance of the breath flow pathway. The apparatus also includes a plurality of sensors positioned in the breath flow pathway, downstream from the breath inlet, wherein each sensor is configured to detect the presence of a biomarker indicative of respiratory disease. The apparatus further includes a breath outlet positioned at an exit of the breath flow pathway, downstream from the sensors. An excess exhaust portal may be located upstream from the plurality of sensors, and the excess exhaust portal is adapted to release a portion of a breath gas sample entering the breath gas inlet without contacting the sensors. In some embodiments, the plurality of sensors may include a CO selective sensor and a CO₂ selective sensor, and in some instances, a heating element is disposed on an upstream side of the CO selective sensor. The plurality of sensors may include a CO₂ selective sensor, a CO selective sensor, and a NO selective sensor. The biomarker may be selected from the group consisting of CO₂, CO, and NO. The plurality of sensors may be selected from the group consisting of a CO₂ selective sensor, a CO selective sensor, and a NO selective sensor.

A method for monitoring respiratory disease is described that includes the steps of flowing a breath gas sample through a breath inlet positioned at an entrance of a housing, and exposing the breath gas sample to a plurality of sensors positioned in the breath flow pathway, downstream from the breath inlet. The housing comprises a breath flow pathway disposed within the housing. Each sensor is configured to detect the presence of a biomarker indicative of respiratory disease. The method further comprises the step of releasing the breath gas sample through a breath outlet positioned at an exit of the breath flow pathway.

In some embodiments, a sensor may include multiple sensor units, each providing one or more signals that may be indicative of the presence or concentration of a particular analyte. The analyte may be detected, or its concentration estimated, based on the signals obtained from the multiple sensor units.

Fewer than the sensors described in the above examples, additional sensors, different combinations, other sensors, or sub-combinations of the described sensors may be used for the detection of respiratory disease. Moreover, the sensors and related components may be positioned in different configurations and breath flow pathways than those described in the above examples.

### BRIEF DESCRIPTION OF THE SEVERAL VIEWS OF THE DRAWINGS

FIGS. 1 through 3 illustrate schematically a device with multiple sensors for the detection of respiratory disease, in accordance with an embodiment of the present invention. FIG. 1 illustrates schematically a perspective view from the back of the device. FIG. 2 illustrates schematically a perspective view from the front of the device. FIG. 3 illustrates schematically a cutaway view of internal components of a device with multiple sensors for the detection of respiratory disease, in accordance with an embodiment of the present invention. The device includes a carbon monoxide (CO) selective sensor, a carbon dioxide (CO₂) selective sensor, and a nitric oxide (NO) selective sensor.

FIG. 4 provides a block diagram of a CO/CO₂ exhalation gas detection unit for COPD and pulmonary disease, in accordance with an embodiment of the present invention. The device includes a CO selective sensor and a CO₂ selective sensor.

FIG. 5 illustrates schematically a cutaway side view of a CO/CO₂ exhalation gas detection unit for COPD and pulmonary disease. The device includes a CO selective sensor module and a CO₂ selective sensor module enclosed in a casing.

### DETAILED DESCRIPTION OF THE INVENTION

As used herein, the terms “comprises,” “comprising,” “includes,” “including,” “has,” “having,” or any other variation thereof, are intended to cover a non-exclusive inclusion. For example, processes, methods, articles, or apparatuses that comprise a list of elements are not necessarily limited to only those elements but may include other elements not expressly listed or inherent to such processes, methods, articles, or apparatuses. Further, unless expressly stated to the contrary, “or” refers to an inclusive “or” but not to an exclusive “or.” For example, a condition A or B is satisfied by any one of the following: A is true (or present) and B is false (or not present), A is false (or not present) and B is true (or present), and both A and B are true (or present).

Also, use of “a” or “an” are employed to describe the elements and components of the invention. This is done merely for convenience and to give a general sense of the invention. This description includes one or at least one and the singular also includes the plural unless it is obvious that it is meant otherwise.

Unless otherwise defined, technical and scientific terms used herein have the same meaning as commonly understood by one of ordinary skill in the art to which this invention belongs. Although methods that are similar or equivalent to those described herein can be used in the practice or testing of the present invention, suitable methods and materials are described herein. All publications, patent applications, patents, and other references mentioned herein are incorporated by reference in their entirety. In case of conflict, the present specification, including definitions, will control. In addition, materials, methods, and examples are illustrative only and not intended to be limiting.

In the following description, numerous specific details, such as the identification of various system components, are provided to understand the embodiments of the invention. One skilled in the art will recognize, however, that embodiments of the invention can be practiced without one or more of the specific details, ordinary methods, components, materials, etc. In still other instances, well-known structures, materials, or operations are not shown or described in detail to avoid obscuring aspects of various embodiments of the invention.

Reference throughout this specification to “one embodiment” or “an embodiment” means that a particular feature, structure, or characteristic described in connection with the embodiment is included in at least one embodiment of the present invention. Thus, the appearance of the phrases “in one embodiment” or “in an embodiment” in various places throughout this specification are not necessarily all referring to the same embodiment. Furthermore, the particular features, structures, or work characteristics may be combined in any suitable manner in one or more embodiments.

In one embodiment, a device may include a series of sensors selected to detect certain diagnostic markers for respiratory disease. For example, a combination of sensors may be used in a single unit for the detection of compounds indicative of respiratory disease, such as carbon monoxide (CO), carbon dioxide (CO₂), and nitric oxide (NO), other biomarkers, or any sub-combinations thereof. The apparatus may be useful in providing information regarding respiratory and lung diseases, including CO₂ narcosis.

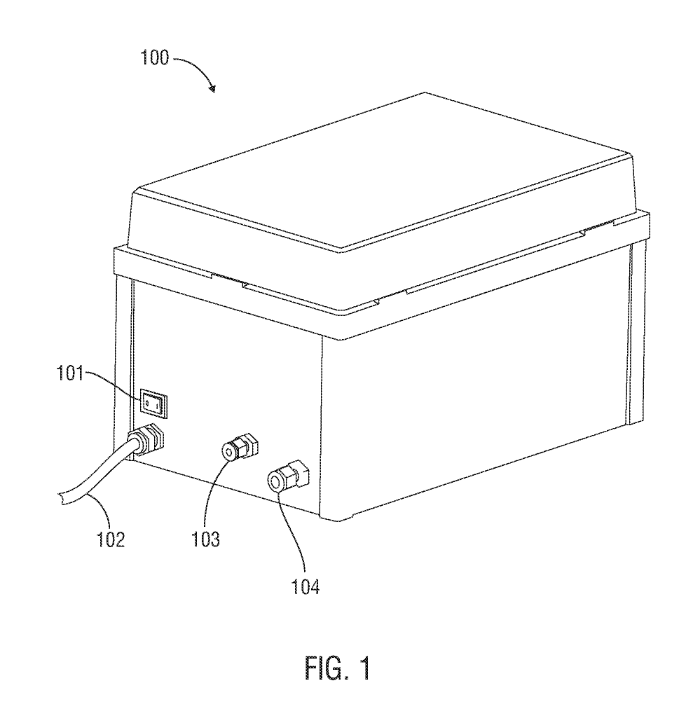

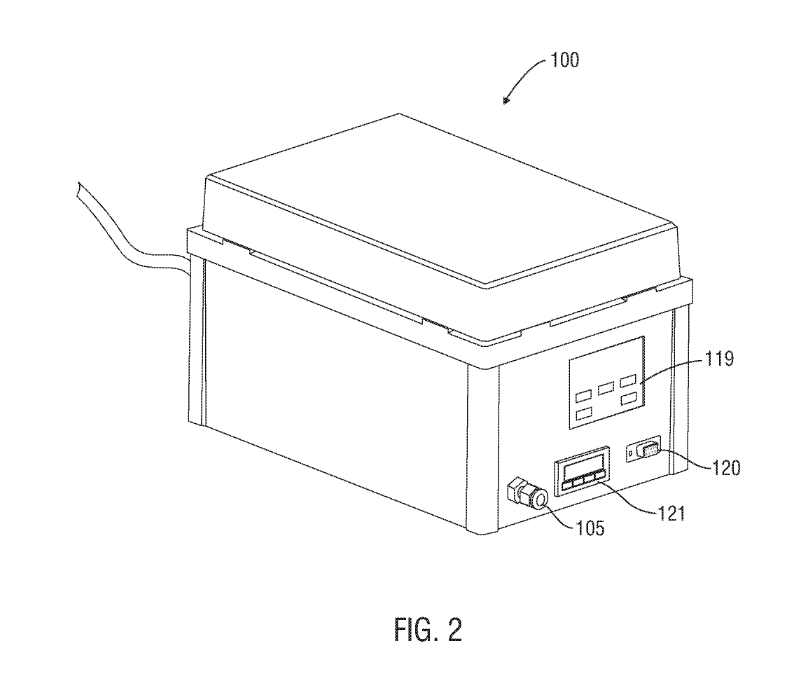

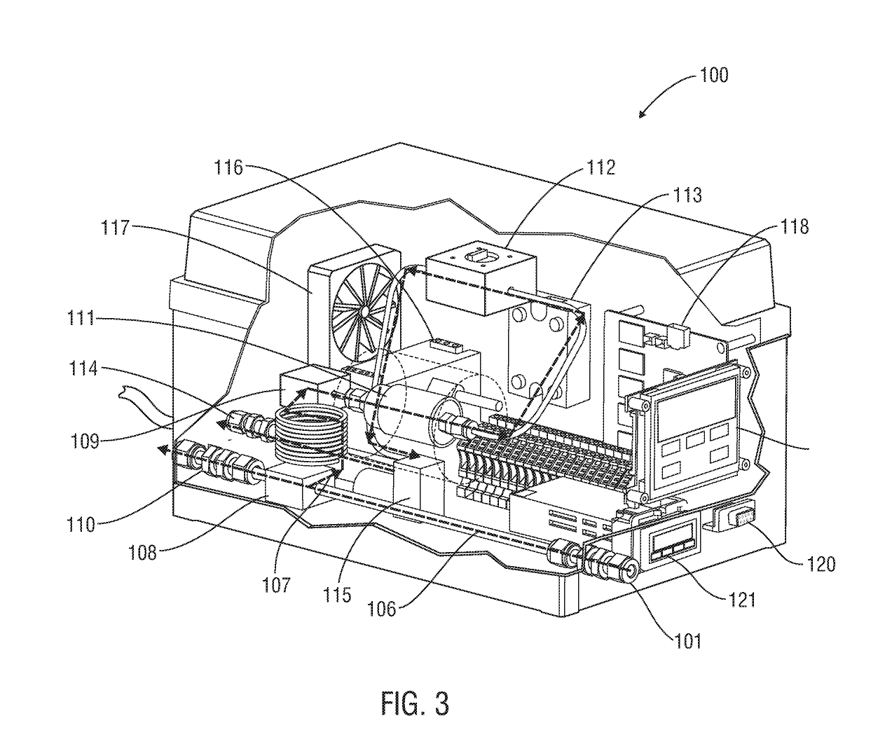

Referring to FIGS. 1 through 3, an apparatus 100 for the detection of respiratory diseases, according to one embodiment of the present invention, is illustrated. As shown in FIG. 1, the device is housed within an enclosure. On one side of the enclosure is a power switch 101, an A/C power cord 102, an outlet for breath exhaust 103, and an excess breath exhaust outlet 104. As shown in FIG. 2, breath gas enters a breath inlet 105 on the other side of the enclosure. As shown in FIG. 3, a portion of breath gas exits the system through a flow pathway 106 that terminates at the excess breath exhaust portal, while the non-exhausted breath gas is channeled through various sensors. In a preferred embodiment, most of the breath gas exits the apparatus through the breath exhaust port. For example, approximately 95% or more of the breath gas volume is exhausted as excess breath, while approximately 5% of less of the breath gas volume proceeds through the apparatus for analysis.

The non-exhausted breath gas is channeled through a breath flow pathway 107 that includes a CO₂ selective sensor 108. The CO₂ selective sensor may be selected from a variety of commercially available sensors known in the art for detecting the presence of the compound in a gas. As a non-limiting example, the CO₂ selective sensor may include spectroscopic nondispersive infrared CO₂ sensor (or NDIR CO₂ sensor), 0.1 W with 5V input, with a detecting range of approximately 1-10% and T60s less than approximately 15 seconds. The descriptions of CO₂ selective sensors are provided as examples for illustrative purposes. Other types of sensors may be used in combination with or instead of this NDIR sensor to detect the presence of CO₂ in a breath gas sample.

The gas proceeds through sensor 109, where the presence of CO may be detected. In some embodiments, the CO selective sensor may be selected from a variety of commercially available sensors known in the art for detecting the presence of the compound in a gas. As a non-limiting example, the CO selective sensor may include a metal oxide semiconductor type sensor, 0.1 W with 3.3V input, with a detecting range of approximately 1-20 ppm, and T60s less than approximately 15 seconds. The descriptions of CO selective sensors are provided as examples for illustrative purposes. Other types of sensors may be used in combination with or instead of a metal oxide semi-conductor type sensor to detect the presence of CO in a breath gas sample. The humidity of the gas may be controlled using one or more humidity controllers 110 to regulate the humidity of the breath gas flowing through the breath flow pathway. As an example, it may be desirable to control the humidity of the gas before it is channeled to and analyzed by a NO sensor assembly, such as that described below.

The breath gas is directed to a NO sensor element. In this embodiment, the breath gas is directed to a micro-channel reactor (MCR) filter 111 and sensor 112 for the detection of NO. A MCR filter heater relay 113 may also be included. The breath gas is then directed through the breath exhaust portal 114 using a pump 115. The MCR and sensor assembly are configured to determine the total NO concentration from the breath sample gas. A patient's breath sample can include nitrogen oxides (NOₓ), which include pure NO, pure nitrogen dioxide (NO₂), and mixtures thereof. The gas introduced from the patient's breath typically has concentrations of NO, NO₂ and CO in the range of 0 to 1000 ppb.

In some embodiments, the MCR filter includes a catalyst filter comprising platinum and zeolite within a flow pathway. The gas flowing through the flow pathway interacts with the catalyst filter at a particular temperature to form an equilibrium mixture of NO and NO₂. The MCR and sensor assembly further includes a sensor element configured to sense the amount of NOₓ flowing therethrough. In a preferred embodiment, the sensor element includes two electrodes on a solid electrolyte yttria-stabilized zirconia as follows: (1) a sensing potentiometric electrode disposed downstream of the catalytic filter device so as to contact the equilibrium mixture of NO and NO₂, and (2) a reference potentiometric electrode. Because the relative amounts of NO and NO₂ are known due to the equilibrium reaction through the filter, the NOₓ reading of the sensor can be used to determine the amount of NO in the sample.

The sensor and the microchannel reactor are maintained at different temperatures to provide a driving force for the NOₓ equilibration reactions. That is, the reactor equilibrates the NO to NO₂ mixture based principally on the temperature of the reactor (which includes platinum-zeolite (PtY)), and then the potential develops on the sensor element based on this equilibration of NO and NO₂ changing when reacting with reference electrode (PtY) and the sensing electrode at a temperature different than the temperature of the reactor. The sensor works by measuring the potential difference between the two electrodes, and a total NOₓ concentration (and then NO concentration) can be calculated by comparing the potential to a calibration curve. Details regarding reactor and sensor assemblies are described in U.S. Patent Publication Nos. 2015-0250408-A1 and 2017-0065208-A1, both entitled “Respiratory Monitor,” the entirety of which are incorporated by reference herein. It will be appreciated that other sensors and sensor assemblies known in the art for detecting NO in the presence of various interference gases also may be used.

The apparatus also may include an A/C DC power supply 116 and a case fan 117 for cooling. Control and acquisition electronics 118, as well as an LCD touch screen 119 that allows a user to enter information may be included, as well. The apparatus may also include external communications output (wired or wireless) 120, along with an Omega temperature controller 121.

The analyte or analytes of interest may be detected, or its concentrations estimated, based on the signals obtained from the sensor units. In some embodiments, information obtained from the sensors may be used to provide qualitative data regarding the respiratory functions of a patient whose breath gas has been analyzed. For example, the measurements obtained from the sensors may be used to determine whether a given patient may exhibit (or not exhibit) certain indicators of asthma or COPD. The information also may be used to obtain quantitative results, such as specific levels of certain gases in a patient's breath.

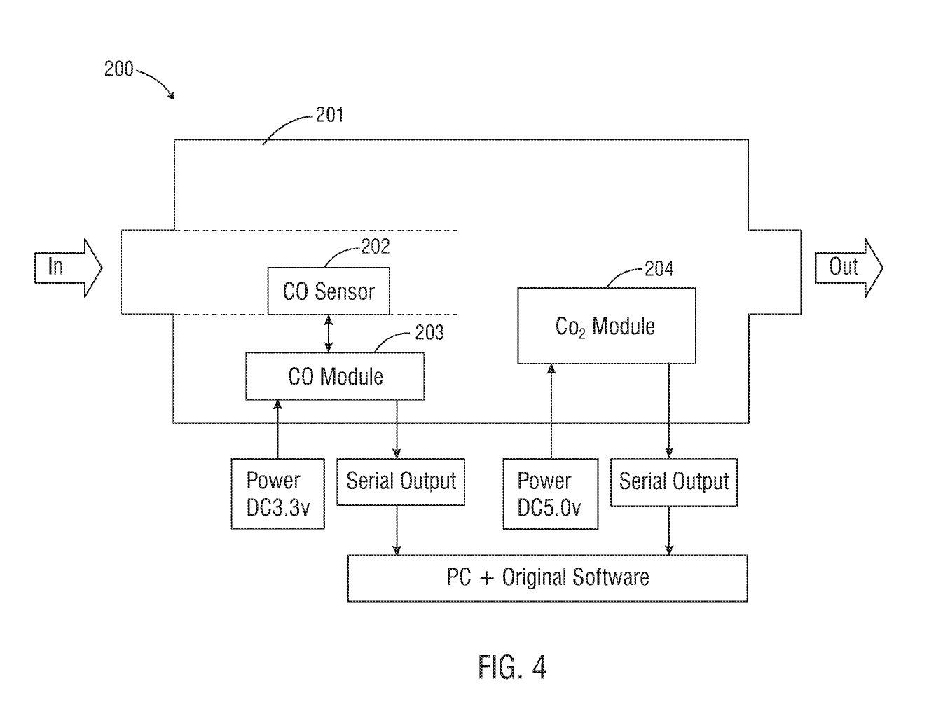

Referring to FIG. 4, a schematic block diagram of a CO/CO₂ exhalation gas detection unit for detecting COPD and pulmonary disease is shown. The unit 200 incorporates a CO and a CO₂ sensor. The sensors may be contained in a casing 201. The casing may be an airtight cylindrical work piece of stainless steel (e.g., SUS 304 or the like); however, other enclosures may be used as well. The CO sensor 202 may be part of a CO sensing module 203, wherein the CO sensor is stretched from the module and placed in the gas flow path inside the detection unit housing. The CO sensor module may be a metal oxide semiconductor type sensor.

For example, the CO sensor may include a CO sensing module FIS 4051-S11 with a supply voltage of 3.3v+/−5%, a detection concentration of approximately 0.3 ppm to approximately 30 ppm, and a serial output signal, which may be procured from Nissha FIS, Inc. The output CO concentrations are 0.00 and approximately 0.3 to 30.0 ppm. The sensor module has a flow measurement of 150 cc per minute and a measurement variation with a coefficient of variation (CV), as determined by the ratio of the standard deviation to the average value, of approximately 10% or less. In a preferred embodiment, the sensor module may have a detection interval time of approximately every one second (every 1 second update of serial output). The time to reach 60% of the saturation point of the sensor signal curve is approximately 90 seconds or less. The temperature is maintained within approximately 10° C. to room temperature. The sensitivity and relative variability (CV) of the sensors have been shown not to be affected or at least minimally affected by the presence of the following gases: acetone (1 ppm), ammonia (NH₃) (1 ppb), CO₂ (8%), ethanol (C₂H₅OH) (300 ppm), hydrogen gas (H₂) (50 ppm), and hydrogen sulfide (H₂S) (25 ppm). The above-described sensors are provided as non-limiting examples, and other CO sensors and CO sensor modules that are known in the art for detecting the CO in a gas sample in the presence of various interference gases may also be used.

A CO₂ sensor also may be positioned in the gas flow path inside the detection unit housing. The sensor may be part of a CO₂ module 204. As a non-limiting example, he unit may include CO₂ module IR-93 (10%) with a Non Dispersive Infrared (NDIR) type sensor with a supply voltage of 5.0v+/5%. The sensor has a detection concentration of approximately 1.00% to 9.99%, and a serial output signal. CO₂ concentration outputs are approximately 0.00% and 1.00% to 9.99%. The sensor module has a flow measurement of 150 cc per minute and a measurement variation with a CV (standard deviation/average value) of approximately 10% or less. The detection interval is every one-second (and the averaged data of time times are updated every 2 seconds). The time to reach 60% of the saturation point of sensor signal curve is approximately 15 seconds or less. It has been shown that the sensitivity and CV are not affected or at least are minimally affected by the presence of the following gases: acetone (1 ppm), NH₃ (1 ppb), CO (75 ppm), C₂H₅OH (300 ppm), H₂ (50 ppm), and H₂S (25 ppm). The sensor is operated within 10° C. to room temperature. Other CO₂ sensors and CO₂ sensor modules that are known in the art for detecting CO₂ in a gas sample in the presence of various interference gases may also be used.

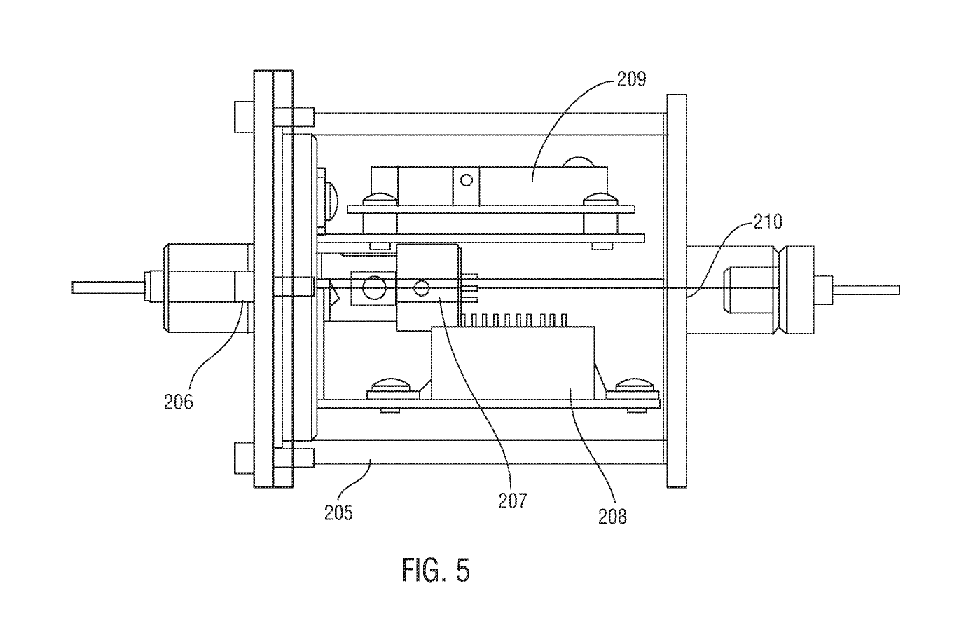

As shown in the example in FIG. 5, the sensors may be positioned within a casing 205 and allowed to contact the breath flow pathway. The gas enters the inlet 206 and contacts the CO sensor 207 from CO module 208 (e.g., CO sensing module FIS4051-S11). Then the gas contacts the CO₂ module 209 (e.g., CO₂ sensing module IR-93 (10%)) and is released from the device through outlet 210. Here, the CO sensor and CO₂ sensors are in fluid communication using flexible tubing (not shown) that is attached to both modules, forming a pathway between CO sensor 207 and CO₂ module 208. The sensor may be connected inside the unit with a relay connector and can be exchanged. In this embodiment, the two sensor modules are fluidly connected together so that gas flows from the CO sensor directly into the CO₂ sensor. The gas then exhausts from there into the (stainless steel) case.

In some instances, the presence of volatile organic compounds (VOC gas) may interfere with the accuracy of the sensor readings, for example the accuracy of the readings for the CO sensor. Thus, in a preferred embodiment, a heating device such as a heating pad or other heating element is provided on the upstream side of one more sensor detection elements, such as the CO sensor. The VOC gas contained in the gas to be measured is burned and removed in order to improve the measurement accuracy for the target gas. For example, a heating device may be positioned upstream of the CO sensor, and preferably at or near the port of entry of a breath gas sample. One or more heating devices may be placed elsewhere within the casing, and multiple heating devices may be incorporated within the device.

It will be appreciated that fewer than the described sensors or additional sensors, different combinations, other sensors, or sub-combinations of the described sensors may be used to monitor medical conditions of interest. For example, a device may include sensors for CO as a smoke analyzer. This sensor may be useful in the prevention of COPD, and it may be used to assess the risk of COPD for an individual. The device may also include one or more CO₂ sensors useful for the monitoring of COPD and lung disease. The detection of CO₂ may be particularly useful for patients undergoing oxygen therapy to treat COPD, as it may provide useful information regarding the risk of CO₂ narcosis.

In another embodiment, a device may include sensors for CO and NO detection. Detection of these compounds may allow a clinician, patient, or other user to distinguish between asthma, COPD, and Asthma and COPD Overlap Syndrome (ACOS) in a single breath test. In yet another embodiment, a device may include sensors for CO₂ and NO detection to allow for diagnosis of respiratory diseases. In yet other embodiments, a device may include sensors for the detection of respiratory disease biomarkers CO₂, CO, and NO in a breath gas sample.

In addition, the sensors and sensor modules may be used in combination with other sensors and sensor modules to test a given breath gas sample for biomarkers associated with multiple medical conditions. For example, the apparatus may include sensors for detecting known biomarkers for respiratory diseases such as CO and CO₂, along with sensors for detecting known biomarkers for hyperglycemia such as ethanol, acetone, methyl nitrate, isoprene, cyclopentane, and 1-methyl-3-(1 methylethyl)-benzene. In this configuration, the apparatus would allow for the detection of respiratory diseases and hyperglycemia from a patient's breath sample using the same apparatus.

The sensors and related components may be positioned in different configurations and breath flow pathways than those described in the above examples. For example, breath gas proceeding through the breath flow pathway may be exposed to the sensors in different sequences from those described in the above examples.

Breath gas proceeding through the breath flow pathway may be exposed to additional sensors, intermediate sensors, mechanical components, intermediate components, and other system components. For example, the system may include additional components to pre-condition or treat a given gas sample before being exposed to a sensor module. In addition, breath gas proceeding through the breath flow pathway may be exposed to additional sensors, intermediate sensors, mechanical components, intermediate components, and other system components through different pathways.

It will be appreciated that each of the analytes indicative of respiratory disease in a breath gas sample is not limited to detection by the device structures described or the devices described in the above examples. That is, any sensor or combination of sensors that are capable of detecting the presence of analytes that are indicative of respiratory disease in a breath sample may be used.

It also will be appreciated that each analyte in a breath gas sample is not limited to detection by a single sensor, and the sensors described above (e.g., a CO₂ selective sensor, a CO selective sensor, a NO selective sensor, or other sensors for respiratory disease) are not limited to single sensor assemblies, each providing a single signal. Rather, in some embodiments, a sensor may include multiple sensor assemblies. Each sensor assembly may provide its own signal or set of signals. The analyte of interest may be detected, or its concentration estimated, from signals obtained from these multiple sensor assemblies.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with anyone or more of the features described herein. The breadth of the present invention is not to be limited by the subject specification, but rather only by the plain meaning of the claim terms employed.

This disclosure is sufficient to enable one of ordinary skill in the art to practice the invention, and provides the best mode of practicing the invention presently contemplated by the inventor. While a full and complete disclosure is made of specific embodiments of this invention, the invention is not limited by the exact construction, dimensional relationships, and operation shown and described. Various modifications, alternative constructions, design options, changes and equivalents will be readily apparent to those skilled in the art and may be employed, as suitable, without departing from the spirit and scope of the invention. Such changes might involve alternative materials, components, structural arrangements, sizes, shapes, forms, functions, operational features and the like.

1. An apparatus for detecting respiratory disease comprising:
(a) a housing comprising a breath flow pathway disposed within the housing;
(b) a breath gas inlet positioned at an entrance of the breath flow pathway;
(c) a carbon monoxide (CO) sensor detection element positioned in the breath flow pathway, downstream from the breath gas inlet;
(d) a carbon dioxide (CO₂) sensor detection element positioned in the breath flow pathway, downstream from the CO sensor detection element; and
(e) a breath outlet positioned at an exit of the breath flow pathway, downstream from the sensor detection elements.

2. The apparatus of claim 1, further comprising a heating element disposed on an upstream side of the CO sensor detection element.

3. The apparatus of claim 1, wherein the CO sensor detection element comprises a metal oxide semiconductor type sensor.

4. The apparatus of claim 3, wherein the CO₂ sensor detection element comprises a spectroscopic nondispersive infrared (NDIR) CO₂ sensor.

5. An apparatus for detecting respiratory disease comprising:
(a) a housing comprising a breath flow pathway disposed within the housing;
(b) a breath gas inlet positioned at an entrance of the breath flow pathway;
(c) a carbon dioxide (CO₂) sensor element positioned in the breath flow pathway, downstream from the breath gas inlet;
(d) a carbon monoxide (CO) sensor element positioned in the breath flow pathway, downstream from the CO₂ sensor element;
(e) a nitric oxide (NO) sensor element positioned in the breath flow pathway, downstream from the CO sensor element; and
(f) a breath outlet positioned at an exit of the breath flow pathway, downstream from the sensor elements.

6. The apparatus of claim 5, wherein the NO sensor element comprises a micro-channel reactor filter comprising platinum and zeolite, and a potentiometric sensor.

7. The apparatus of claim 6, wherein the NO sensor element further comprises a micro-channel reactor filter heater relay.

8. The apparatus of claim 5, further comprising a humidity controller configured to regulate the humidity of a breath sample in at least a portion of the breath flow pathway.

9. The apparatus of claim 5, further comprising an excess exhaust portal adapted to release from the housing a portion of a breath gas sample entering the breath gas inlet while another portion of the breath gas sample proceeds to the CO₂ sensor element.

10. A multi-sensor apparatus for monitoring respiratory disease comprising:
(a) a housing comprising a breath flow pathway disposed within the housing;
(b) a breath inlet positioned at an entrance of the breath flow pathway;
(c) a plurality of sensors positioned in the breath flow pathway, downstream from the breath inlet, wherein each sensor is configured to detect the presence of a biomarker indicative of respiratory disease; and
(d) a breath outlet positioned at an exit of the breath flow pathway, downstream from the sensors.

11. The apparatus of claim 10, further comprising an exhaust portal adapted to release a portion of a breath gas sample entering the breath inlet without contacting the sensors.

12. The apparatus of claim 10, wherein the plurality of sensors comprises a carbon monoxide (CO) selective sensor and a carbon dioxide (CO₂) selective sensor.

13. The apparatus of claim 12, further comprising a heating element disposed on an upstream side of the CO selective sensor.

14. The apparatus of claim 12, wherein the CO selective sensor comprises a metal oxide semiconductor type sensor.

15. The apparatus of claim 12, wherein the CO₂ selective sensor comprises a spectroscopic nondispersive infrared (NDIR) CO₂ sensor.

16. The apparatus of claim 10, wherein the plurality of sensors comprises a carbon dioxide (CO₂) selective sensor, a carbon monoxide (CO) selective sensor, and a nitric oxide (NO) selective sensor.

17. The apparatus of claim 10, wherein the biomarker is selected from the group consisting of carbon dioxide (CO₂), carbon monoxide (CO), and nitric oxide (NO).

18. The apparatus of claim 10, wherein the plurality of sensors is selected from the group consisting of a carbon dioxide (CO₂) selective sensor, a carbon monoxide (CO) selective sensor, and a nitric oxide (NO) selective sensor.

19. A method for detecting respiratory disease comprising:
(a) flowing a breath gas sample through a breath inlet positioned at an entrance of a housing, wherein the housing comprises a breath flow pathway disposed within the housing;
(b) exposing the breath gas sample to a carbon dioxide (CO₂) sensor detection element positioned in the breath flow pathway, downstream from the breath inlet;
(c) exposing the breath gas sample to a carbon monoxide (CO) sensor detection element positioned in the breath flow pathway, downstream from the CO₂ sensor detection element; and
(d) releasing the breath gas sample through a breath outlet positioned at an exit of the breath flow pathway.

20. The method of claim 19, further comprising the step of heating the breath gas sample using a heating element disposed on an upstream side of the CO sensor detection element.

21. The method of claim 19, wherein the CO sensor detection element comprises a metal oxide semiconductor type sensor.

22. The method of claim 21, wherein the CO₂ sensor detection element comprises a spectroscopic nondispersive infrared (NDIR) CO₂ sensor.
